(19) United States
(12) Patent Application Publication
(10) Pub. No.: US 2015/0135437 A1
(43) Pub. Date: May 21, 2015

(54) PATIENT TABLE, AND METHOD AND APPARATUS FOR COMPENSATING SAGGING OF PALLET OF PATIENT TABLE

(71) Applicant: Shenyang Neusoft Medical Systems Co., Ltd., Shenyang (CN)

(72) Inventors: Shuangxue LI, Shenyang (CN); Hong YAN, Shenyang (CN)

(21) Appl. No.: 14/513,220

(22) Filed: Oct. 14, 2014

(30) Foreign Application Priority Data
Nov. 21, 2013 (CN) 201310598173.3

(51) Int. Cl. A61B 6/00; A61B 6/04

(52) U.S. Cl. CPC A61B 6/5276; A61B 6/0407
USPC 5/601

(57) ABSTRACT

A method for compensating the sagging of a pallet of the patient table includes: detecting a degree of sagging of a position on the pallet where a scanning cross section is located; determining whether the degree of sagging is smaller than a predetermined degree, and ending the procedure if the degree of sagging is smaller than the predetermined degree; otherwise, determining an adjustment amount of the pallet according to the degree of sagging, and lifting the pallet according to the adjustment amount. An apparatus includes a detecting device; a lifting device; and a controller. If a detected value of the degree of sagging is smaller than a predetermined value, the controller ends the procedure; otherwise, the controller is configured to determine an adjustment amount of the pallet according to the degree of sagging, and control the lifting device to lift the pallet according to the adjustment amount.

The present application claims the benefit of priority to Chinese Patent Application No. 201310598173.3, titled “PATIENT TABLE, AND METHOD AND APPARATUS FOR COMPENSATING SAGGING OF PALLET OF PATIENT TABLE”, filed with the Chinese State Intellectual Property Office on Nov. 21, 2013, the entire disclosure of which is incorporated herein by reference.

### TECHNICAL FIELD

The present application relates to the technical field of medical equipment, and particularly to a method for compensating sagging of a pallet of a patient table. The present application further relates to an apparatus using the above method, and a patient table having the apparatus.

### BACKGROUND

Currently, a medical computed tomography (CT) machine is a fully functioning apparatus for detecting a condition of a patient, and computed tomography is short for computed X-ray cross-sectional scanning technique, which performs cross-sectional examination of the human body with X-ray.

An existing medical CT machine generally includes a scanner, a patient table, a console, and etc . . . The patient table includes a frame for supporting and a pallet arranged on the frame, and the pallet and the frame are horizontally slidably connected with respect to each other, which may facilitate the sliding of the pallet out of and into the frame.

Before performing the CT scanning, the patient is required to lie on the pallet of the patient table; then, the pallet slides outwardly with respect to the frame, and is supported on the frame with its tail end, and a head end of the pallet extends into the scanner along with the main body of the pallet, thereby sending the human body into the scanner to be scanned.

In the scanning process, since the pallet extends by a great length, and the patient applies a downward force to the pallet continuously, thus the pallet sags, and a scanning cross section on the pallet sags accordingly. However, when the CT machine is used together with other equipment (e.g., Positron Emission Tomography), the scanning cross section is required to be repositioned, while the sagging of the pallet negatively affects the accuracy of repositioning to a large extent.

Thus, the sagging of the pallet causes a difference between a practical scanning cross section and a theoretical scanning cross section (a cross section to be scanned when the pallet does not sag), and such difference negatively affects the accuracy of repositioning, which causes inconvenience for the following treatments.

Therefore, a technical problem to be solved presently by those skilled in the art is to design a method and an apparatus for compensating the sagging of the pallet of the patient table, which may unify the practical scanning cross section with the theoretical scanning cross section to improve the accuracy of positioning.

### SUMMARY

A method and an apparatus for compensating sagging of a pallet of a patient table are provided according to the present application, which may unify a practical scanning cross section and a theoretical scanning cross section to improve the accuracy of positioning.

A patient table is further provided according to the present application, which may compensate sagging of a pallet of the patient table.

To address the above technical issues, a method for compensating sagging of a pallet of a patient table is provided according to the present application, which includes the following steps:

11) detecting a degree of sagging of a position on the pallet where a scanning cross section is located;

12) determining whether the degree of sagging is smaller than a predetermined degree, and ending the procedure in a case that the degree of sagging is smaller than the predetermined degree; or proceeding to step 13) in a case that the degree of sagging is not smaller than the predetermined degree; and

13) determining an adjustment amount of the pallet according to the degree of sagging, and lifting the pallet according to the adjustment amount.

In the method according to the present application, the degree of sagging of the pallet is first detected, and if the degree of sagging is small, no substantial effect will be caused to the positioning, thus for simplifying the procedure, the procedure may be ended directly without compensating the sagging of the pallet. If the degree of sagging is large, the adjustment amount may be determined according to the degree of sagging of the pallet, and then the pallet is lifted according to the adjustment amount so as to lift the position on the pallet corresponding to the scanning cross section above the theoretical position. When a human body lies on the pallet, the pallet sags under the effect of the gravity of the human body, which lowers the scanning cross section to the height of the theoretical position, thus the scanning cross section practically detected is just the theoretical scanning cross section, thereby improving the accuracy of the positioning.

Preferably, the degree of sagging has a linear relationship with an elastic coefficient K of the pallet per unit weight, a distance L between a pallet front support and a tail portion of the pallet, a distance S between the scanning cross section and the pallet front support, and a weight G of a patient, and in the step 13), the adjustment amount X is obtained according to the following formula:

X=K×L×S×G/(L+S).

Before performing the compensation of the sagging of the pallet, it is critical to calculate the adjustment amount. According to research, the adjustment amount has a linear relationship with the elastic coefficient K of the pallet per unit weight, the distance L between the pallet front support and the tail portion of the pallet, the distance S between the scanning cross section and the pallet front support, and the weight G of the patient, and these variables may be read by the scanner directly, thus the adjustment amount of the pallet may be calculated without measuring again, which further simplifies the adjustment process.

Preferably, the step 13) includes:

131) calculating the adjustment amount of the pallet according to the degree of sagging; and

132) determining whether the adjustment amount exceeds a predetermined range, and lifting the pallet front support of the upper frame according to the adjustment amount in a case that the adjustment amount does not exceed the predetermined range, or moving the upper frame upwardly and recalculating the adjustment amount in a case that the adjustment amount exceeds the predetermined range, till the adjustment amount is within the predetermined range, and lifting the pallet front support according to a difference value between the recalculated adjustment amount and an upward displacement of the upper frame.

In a case that the adjustment amount of the pallet is large, by only lifting the pallet may not meet the requirement for compensation, here, the upper frame may be lifted first to lift the whole pallet to a certain height, then the height of the pallet may be adjusted slightly by lifting the pallet front support, thereby ensuring the positioning accuracy of the scanning cross section.

Preferably, in the step 132), the upper frame is hinged to a top end of the frame, first, the frame is swung forwardly to push the upper frame to move forward and upward, then the pallet is moved rearward according to a forward displacement of the upper frame, and the distance between the pallet front support and the tail portion of the pallet and the distance between the scanning cross section and the pallet front support are re-determined to recalculate the adjustment amount.

An apparatus for compensating sagging of a pallet of a patient table is further provided according to the present application, which includes a detecting device configured to detect a degree of sagging of a position on the pallet where a scanning cross section is located; a lifting device configured to lift or lower the pallet; and a controller which is in signal connection with both of the detecting device and the lifting device. The controller is pre-stored with a predetermined value of the degree of sagging, and in a case that a detected value of the degree of sagging is smaller than the predetermined value, the controller is configured to end the procedure; and in a case that the detected value of the degree of sagging is not smaller than the predetermined value, the controller is configured to determine an adjustment amount of the pallet according to the detected value of the degree of sagging, and then control the lifting device to lift the pallet according to the adjustment amount.

Preferably, the apparatus further includes a frame having an upper frame, wherein the pallet is slidably connected to the upper frame, and after the pallet slides to a position corresponding to the scanning cross section, a tail portion of the pallet is fixedly connected to the upper frame; and the lifting device includes a pallet front support configured to support an extended portion of the pallet, and the pallet front support is movable up and down.

The pallet front support is movable up and down to move the pallet upward or downward by a certain height, thereby adjusting the height position of the pallet, and finally, positioning the scanning cross section at a required height. The whole adjustment process is simple and the accuracy of the positioning is high.

Preferably, the pallet front support is a carrier roller supported on the upper frame with its two ends, and the carrier roller is rollable up and down.

Preferably, the lifting device further includes a link mechanism connected to a bottom end of the upper frame, and the link mechanism is swingable in a front-rear direction to drive the upper frame to move in a frontward and upward direction or a rearward and downward direction.

Preferably, the frame has a base seat, the lifting device further includes two connecting plates connected between the upper frame and the base seat, and the two connecting plates extend right and left and are parallel to each other, each of the connecting plate has two ends hinged to the upper frame and the base seat respectively, and hinged shafts extend right and left and are parallel to each other.

The swinging of the upper frame may be realized by the swinging of the link mechanism or the connecting plates, which may change the height position of the upper frame, thereby lifting or lowering the pallet to position it at the required position, and realizing the compensation of the sagging of the pallet. Further, the link mechanism or the connecting plates may cooperate with the pallet front support to improve the adjustment efficiency and positioning accuracy.

A patient table is further provided according to the present application, which has the apparatus according to any one of the above solutions. Thus the patient table of the present application also has the technical effects generated by the apparatus according to any one of the above solutions, which will not be described herein.

### BRIEF DESCRIPTION OF THE DRAWINGS

FIG. 1 is a schematic flow chart of a method according to an embodiment of the present application;

FIG. 2 is a structural diagram of the pallet supporting in an ideal state;

FIG. 3 is a structural diagram of the pallet supporting in a practical state;

FIG. 4 is a structural diagram of the pallet supporting after being compensated with the method of the present application;

FIG. 5 is a schematic perspective view showing the structure of a patient table according to the present application in a state that the pallet thereof does not extend out of the patient table;

FIG. 6 is a schematic view of the patient table shown in FIG. 5 in a state that the pallet thereof extends out of the patient table;

FIG. 7 is a schematic view of the patient table shown in FIG. 5 in a state that an upper frame thereof is lifted; and

FIG. 8 is a schematic view of the patient table shown in FIG. 5 in a state that the upper frame thereof is lowered.

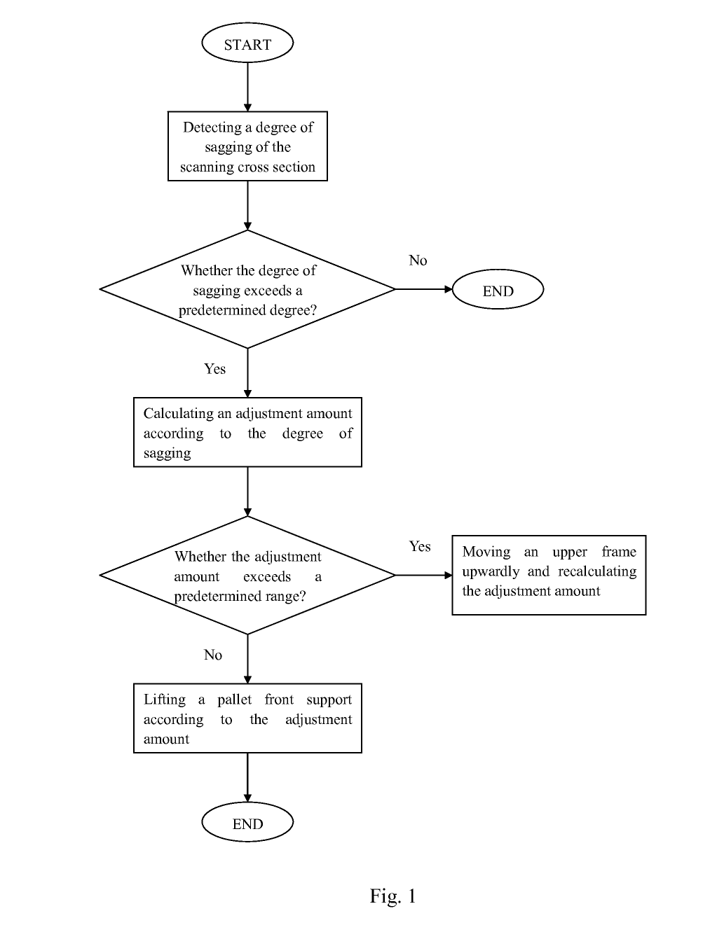

Reference Numerals in FIGS. 1 to 8:

| | | | | | |
|---|---|---|---|---|---|
| 1 | pallet, | 2 | frame, | 21 | upper frame, |
| 211 | pallet front support; | 22 | base seat; and | 3 | connecting plate. |

### DETAILED DESCRIPTION

A method and an apparatus for compensating the sagging of a pallet of a patient table are provided according to the present application, which may unify a practical scanning cross section and a theoretical scanning cross section to improve the accuracy of positioning.

A patient table is further provided according to the present application, which may compensate the sagging of the pallet.

For those skilled in the art to better understand technical solutions of the present application, the present application is described in detail in conjunction with drawings and embodiments hereinafter.

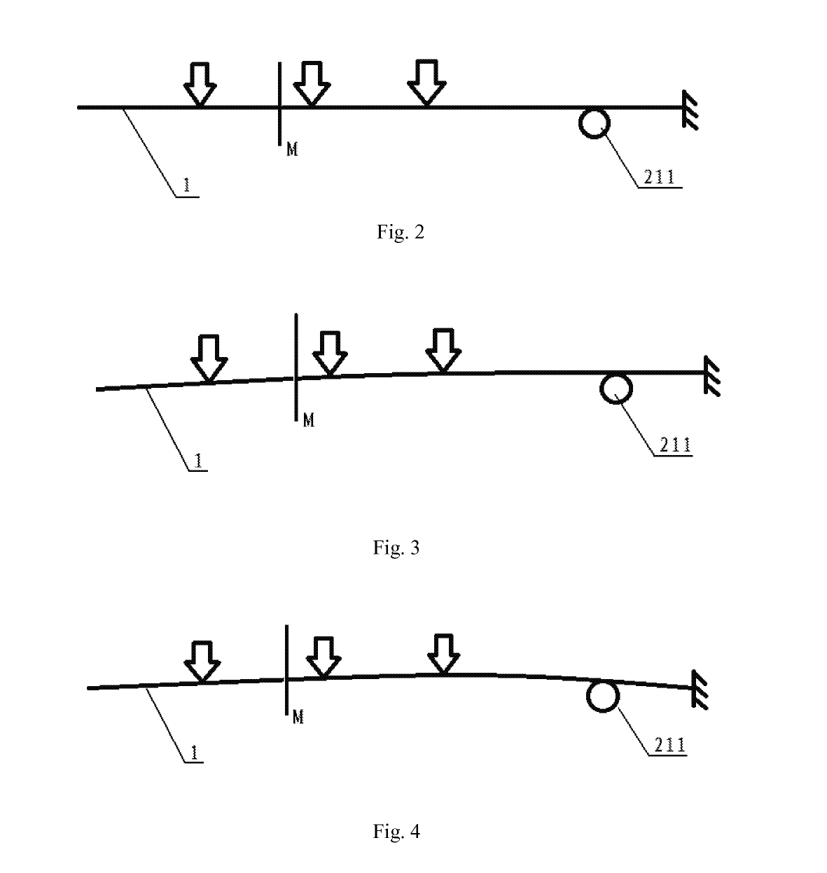

Reference is made to FIGS. 1 to 4. FIG. 1 is a schematic flow chart of a method according to an embodiment of the present application; FIG. 2 is a structural diagram of the pallet supporting in an ideal state; FIG. 3 is a structural diagram of the pallet supporting in a practical state; and FIG. 4 is a structural diagram of the pallet supporting after being compensated with the method of the present application.

Terms indicating directions and positions herein, such as “front”, “rear”, “up”, “down”, “left”, “right”, are defined by taking the patient table as a reference. Front refers to a direction in which the patient table moves towards the scanner, and accordingly, rear refers to a direction in which the patient table moves out of the scanner; down refers to a direction in which the patient table moves close to the ground, and up refers to a direction in which the patient table moves away from the ground; and left and right refer to directions perpendicular to front and rear in a plane where the patient table is located.

To compensate the sagging of the pallet of the patient table, the method of the present application includes the following steps.

Step S11 may include detecting a degree of sagging of a position on the pallet where a scanning cross section is located. In scanning, a portion of the body of a patient to be scanned is required to be sent into a scanner, a plane where the portion to be scanned is located is the scanning cross section, and the position where the scanning cross section is located refers to the position on the pallet corresponding to the portion to be scanned. The degree of sagging may be a sagging height or a sagging angle of the scanning cross section.

Step S12 may include comparing a detected degree of sagging with a predetermined allowable degree of sagging of the pallet, determining whether the detected degree of sagging is smaller than the predetermined degree, and ending the procedure in a case that the detected degree of sagging is smaller than the predetermined degree, which means that a sagging amount of the pallet is within an allowable range, or proceeding to step S13 in a case that the detected degree of sagging is not smaller than the predetermined degree, which means that the sagging amount of the pallet is required to be compensated.

Step S13 may include determining an adjustment amount X required for compensating the sagging of the pallet according to the detected degree of sagging.

Step S14 may include comparing the adjustment amount X with a predetermined range, determining whether the adjustment amount X exceeds the predetermined range, and proceeding to step S15 in a case that the adjustment amount X exceeds the predetermined range; or proceeding to step S16 in a case that the adjustment amount X does not exceed the predetermined range.

Step S15 may include moving an upper frame upwardly, then recalculating the adjustment amount X, and proceeding to the step S14.

Step S16 may include lifting a pallet front support of the upper frame according to the adjustment amount X, and further lifting the pallet till the position on the pallet where the scanning cross section is located approximately coincides with the position of a theoretical scanning cross section.

The step S16 may further include proceeding to the step S12, to further determine whether the degree of sagging after being adjusted is within the predetermined allowable degree, and ending the procedure in a case that the degree of sagging after being adjusted is within the predetermined allowable degree; otherwise, repeating the above compensation procedure.

It is to be noted that, in a case that the adjustment amount X is within the predetermined range after the adjustment amount X is recalculated in the step S15, the lifting amount of the upper frame is required to be subtracted when executing the step S16, since the upper frame is lifted, that is, the pallet front support is lifted according to a value obtained by subtracting the upward movement amount of the upper frame from the recalculated adjustment amount X.

According to the research, the adjustment amount X is related to the degree of sagging of the pallet, and main factors that determine the degree of sagging of the pallet include the elasticity of the pallet, the weight of a patient, and a distance between the scanning cross section and the pallet front support.

Assuming that an elasticity coefficient of the pallet per unit weight is K, and after the pallet extends out of the patient table, a distance between the pallet front support and a tail portion of the pallet is L, a distance between the scanning cross section and the pallet front support is S, and the weight of the patient is G Under normal condition, there is a linear relationship between the adjustment amount X and the variables K, L, S, G In the step S13, the adjustment amount X may be obtained according to the following formula:

X=K×L×S×G/(L+S).

It should be understood by the person skilled in the art that, when the force applied to the pallet is within a small range, the deformation of the pallet is in a linear state substantially, and when the force applied to the pallet is further increased, the deformation of the pallet tends to be aggravated, and the deformation in this case is in a curve state substantially, that is, the deformation line is not an approximately linear straight line, but an arc-shaped curve. Thus, the linear relationship between the adjustment amount X and the variables K, L, S, G is changed accordingly, and the value calculated by directly employing the above formula may not be accurate.

Therefore, in the step S14, the calculated adjustment amount X may be compared with the predetermined range, and the predetermined range refers to the range of the adjustment amount X of the pallet in the linear deformation state. When the adjustment amount X exceeds the predetermined range, which means that the deformation is large, the upper frame may be lifted for pre-compensation, and then the adjustment amount X is re-determined till it is within the predetermined range.

In the step S15, according to an arrangement manner, the upper frame may be hinged to a top end of the frame, and the frame may be swung forwardly, and a supporting member supported at a bottom end of the upper frame swings accordingly. The supporting member of the upper frame is inclined rearwards under normal condition, thus when the frame swings forwardly, the supporting member of the upper frame may push the upper frame to move in a forward and upward direction. To ensure that the position of the scanning cross section is not changed in the front-rear direction, the pallet may be moved rearward, and a rearward displacement of the pallet is coincident with a forward displacement of the upper frame. When the pallet is moved rearwards, the distance L between the pallet front support and the tail portion of the pallet and the distance S between the scanning cross section and the pallet front support are changed accordingly, and at this time, the values of the distances L and S may be re-determined, and the adjustment amount X may be recalculated.

In the Step S11, by taking the plane where the scanning cross section is located in a theoretical state as a theoretical scanning cross section, a distance between the scanning cross section in a sagging state and the theoretical scanning cross section may be detected as a sagging height; or, by taking the ground as a reference, a distance between the scanning cross section in the theoretical state and the ground and a distance between the scanning cross section in the sagging state and the ground may be detected, and the degree of sagging of the scanning cross section may be obtained by comparing the above two distances; and an angle between the pallet before sagging and the pallet after sagging may also be detected.

It is conceivable that, the step S12 may be omitted, the compensation of the sagging of the pallet may be performed regardless of the degree of sagging of the pallet, so as to improve the positioning accuracy of the scanning cross section. Similarly, the step S14 and the step S15 may also be omitted, the compensation may be performed by directly lifting the pallet front support even if the degree of sagging of the pallet is large and the adjustment amount exceeds the predetermined range, only the compensation effect is limited. After the steps S14 and S15 are added, more sagging amount may be compensated effectively by lifting the upper frame, thereby having a good compensation effect.

It is also conceivable that, in the step S13, there may be various methods for calculating the adjustment amount X. For example, in a case that the degree of sagging is measured with the sagging height, the adjustment amount X may directly employ the value of the sagging height, or the adjustment amount for the pallet may be corrected according to the position of the scanning cross section on the pallet. In a case that the degree of sagging is measured with the sagging angle, the adjustment amount X of the pallet may be determined according to the position of the scanning cross section on the pallet in the front-rear direction.

Alternatively, the adjustment amount X of the pallet may be obtained by direct measuring manner. For example, the sagging height of the pallet may be measured by laser ranging as the adjustment amount X of the pallet.

Further, in the step S16, the manner for lifting the pallet is not limited to lifting the pallet front support. Since the pallet front support has a relatively flexible structure and the scanning cross section is in front of the pallet, lifting the pallet front support may effectively adjust the position of the scanning cross section. However, it should be understood by the person skilled in the art that, in the present application, the manner for lifting the pallet is not limited to the above manner, and any manner capable of lifting the plate-shaped structure may be applied to the pallet of the present application to form the lifting device of the present application.

As shown in FIGS. 2 to 4, the weight of the human body applied on the pallet is indicated by an arrow, and the position of the scanning cross section is indicated by the position of a vertical line M. In an ideal state, the pallet is horizontal, and the height of the scanning cross section is coincident with the height of the pallet, as shown in FIG. 2. In practice, the pallet will sag after the patient lies on the pallet, which may cause reduction of the height of the scanning cross section, as shown in FIG. 3, that is, the height of the scanning cross section is lower than the height of the pallet. After the compensation is performed according to the method of the present application, since the pallet front support is lifted, the height of the scanning cross section in an unloaded state is increased, and after the patient lies on the pallet, the pallet sags, thus the scanning cross section may be substantially in the ideal state as shown in FIG. 2, and the practical detected scanning cross section may be coincident with the cross section in the ideal state, thereby facilitating the following diagnoses and treatments.

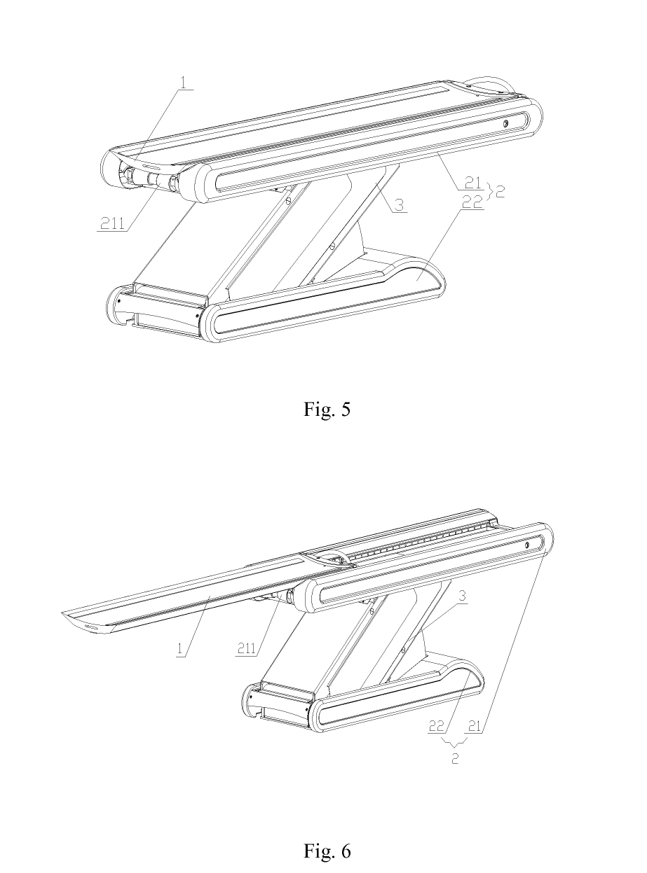

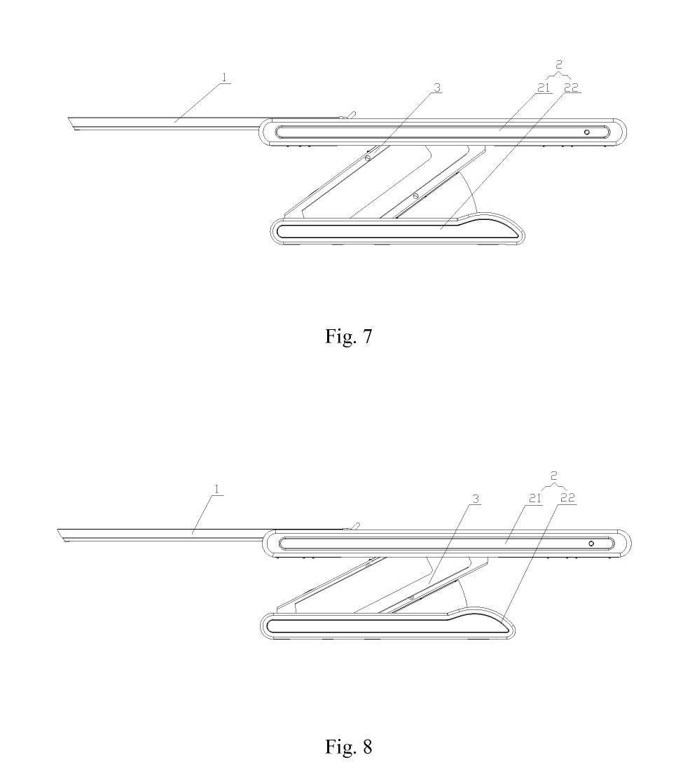

Reference is further made to FIGS. 5 to 8. FIG. 5 is a schematic perspective view showing the structure of the patient table according to the present application in a state that the pallet thereof does not extend out of the patient table; FIG. 6 is a schematic view of the patient table shown in FIG. 5 in a state that the pallet thereof extends out of the patient table; FIG. 7 is a schematic view of the patient table shown in FIG. 5 in a state that the upper frame thereof is lifted; and FIG. 8 is a schematic view of the patient table shown in FIG. 5 in a state that the upper frame thereof is lowered.

An apparatus for compensating the sagging of a pallet of a patient table is further provided according to the present application, which includes a detecting device configured to detect a degree of sagging at a position of a scanning cross section on a pallet 1; a lifting device configured to lift or lower the pallet 1; and a controller which is in signal connection with both of the detecting device and the lifting device. The controller is pre-stored with a predetermined value of the degree of sagging, and is configured to compare a detected value of the degree of sagging detected by the detecting device with the predetermined value. In a case that the detected value of the degree of sagging is smaller than the predetermined value, the controller is configured to end the process; otherwise, the controller is configured to determine an adjustment amount X of the pallet 1 according to the degree of sagging detected by the detecting device, and then control the lifting device to lift the pallet 1 according to the adjustment amount X, so as to lift the scanning cross section to a theoretical height.

The theoretical height refers to a height of the scanning cross section in a case that the pallet 1 is not sagging.

The apparatus according to the present application may further include a frame 2. The frame 2 has an upper frame 21, and the pallet 1 is slidably connected to the upper frame 21, and is slidable rearward and forward with respect to the upper flame 21. When the pallet 1 slides to a position corresponding to the scanning cross section, a tail portion of the pallet 1 is fixedly connected to the upper frame 21 to fix the position of the pallet 1. A front end of the upper frame 21 is provided with a pallet front support 211, and the pallet front support 211 is configured to support an extended portion of the pallet 1 after the pallet 1 extends out, as shown in FIGS. 5 and 6. The pallet front support 211 is movable up and down, thereby forming a part of the lifting device to lift or lower the pallet.

The pallet front support 211 may be a carrier roller. As shown in FIG. 5, the carrier roller is supported on the upper frame 21 with its two ends, and a right end and a left end of the upper frame 21 are both provided with a rolling track for the carrier roller, thus the carrier roller may roll upward and downward to change a height of the supporting position of the pallet 1, thereby adjusting the height of the pallet 1.

The pallet front support 211 may also be a sliding-type supporting plate which is slidable in the up-down direction to lift the pallet 1.

The lifting device may further include a link mechanism. The link mechanism is arranged at a bottom end of the upper frame 21, and is swingable in the front-rear direction. Under normal conditions, the link mechanism is arranged to extend in the up-down direction, and when the link mechanism swings rearward and forward, a height of the link mechanism is changed accordingly, thereby moving the upper frame 21 in a frontward and upward direction or a rearward and downward direction, and in turn pushing the pallet 1 on the upper frame 21 to be lifted or lowered.

Particularly, in a case that the pallet 1 requires a large adjustment amount, the link mechanism may be used to lift the pallet 1 to a large degree, which may reduces the height of the pallet front support required to be lifted, thus the pallet front support 211 may cooperate with the link mechanism to lift the pallet 1 together.

The frame 2 may further be provided with a base seat 22, and the lifting device may further include two connecting plates 3 connected between the upper frame 21 and the base seat 22. The two connecting plates 3 are arranged to be parallel to each other, and to extend right and left, each of the two connecting plates 3 has two ends hinged to the upper frame 21 and the base seat 22 respectively, and hinged shafts extend right and left and are parallel to each other, thereby forming the structure as shown in FIGS. 7 and 8.

Since the connecting plates 3 are hinged between the upper frame 21 and the base seat 22 and are parallel to each other, the whole patient table forms a parallelogram-shaped structure in a case that the upper frame 21 and the base seat 22 are arranged in parallel. Thus, the connecting plates 3 may be swung rearward and forward to push the upper frame 21 to move in the frontward and upward direction or the rearward and downward direction, so as to push the pallet 1 on the upper frame 21 to move upward or downward, as shown in FIGS. 7 and 8.

Apparently, the structure of the connecting plates 3 is similar to the structure of the link mechanism. The upper frame 21 moves in the front-rear direction in addition to the up-down direction, thus the pallet 1 is required to be moved rearward after the pallet 1 is lifted with the above method, so as to move the scanning cross section to the original position in the front-rear direction.

It is to be noted that, the detecting device may be any device capable of detecting the degree of sagging. For example, a laser detector may be used to detect the sagging height, the sagging angel or the like, and alternatively, relevant data stored in the scanner may be used to measure the degree of sagging, without separately providing a corresponding detecting device. The calculation method may be referred to the calculation of the adjustment amount X described hereinbefore, which will not be described herein.

Meanwhile, the lifting device may have various structure forms, which are not limited to the pallet front support 211, the link mechanism and the connecting plates 3. Other components may be employed to lift or lower the pallet 1, for example, a hoist cylinder, an air cylinder or the like.

A patient table is further provided according to the present application, which has an apparatus for compensating the sagging of the pallet of the patient table according to any one of the embodiments described hereinabove, thus the patient table of the present application also has the technical effects generated by the apparatus according to any one of the embodiments described hereinabove, which will not be described herein.

A patient table, a method and an apparatus for compensating the sagging of a pallet of the patient table are described in detail hereinbefore. The principle and the embodiments of the present application are illustrated herein by specific examples. The above description of examples is only intended to help the understanding of the idea of the present application. It should be noted that, for the person skilled in the art, a few of modifications and improvements may be made to the present application without departing from the principle of the present application, and these modifications and improvements are also deemed to fall into the scope of the present application defined by the claims.

1. A method for compensating sagging of a pallet of a patient table, comprising the following steps:
11) detecting a degree of sagging of a position on the pallet where a scanning cross section is located;
12) determining whether the degree of sagging is smaller than a predetermined degree, and ending the procedure in a case that the degree of sagging is smaller than the predetermined degree; or proceeding to step 13) in a case that the degree of sagging is not smaller than the predetermined degree; and
13) determining an adjustment amount of the pallet according to the degree of sagging, and lifting a pallet front support of an upper frame to lift the pallet according to the adjustment amount.

2. The method according to claim 1, wherein the degree of sagging has a linear relationship with an elastic coefficient (K) of the pallet per unit weight, a distance (L) between the pallet front support and a tail portion of the pallet, a distance (S) between the scanning cross section and the pallet front support, and a weight (G) of a patient, and in the step 13), the adjustment amount (X) is obtained according to the following formula:
X=K×L×S×G/(L+S).

3. The method according to claim 2, wherein the step 13) comprises:
131) calculating the adjustment amount (X) of the pallet according to the degree of sagging; and
132) determining whether the adjustment amount (X) exceeds a predetermined range, and lifting the pallet front support of the upper frame according to the adjustment amount (X) in a case that the adjustment amount (X) does not exceed the predetermined range, or moving the upper frame upwardly and recalculating the adjustment amount (X) in a case that the adjustment amount (X) exceeds the predetermined range, till the adjustment amount (X) is within the predetermined range, and lifting the pallet front support according to a difference value between the recalculated adjustment amount (X) and an upward displacement of the upper frame.

4. The method according to claim 3, wherein in the step 132), the upper frame is hinged to a top end of the frame, first, the frame is swung forwardly to push the upper frame to move forward and upward, then the pallet is moved rearward according to a forward displacement of the upper frame, and the distance (L) between the pallet front support and the tail portion of the pallet and the distance (S) between the scanning cross section and the pallet front support are re-determined to recalculate the adjustment amount (X).

5. An apparatus for compensating sagging of a pallet of a patient table, comprising a detecting device configured to detect a degree of sagging of a position on the pallet where a scanning cross section is located; a lifting device configured to lift or lower the pallet; and a controller which is in signal connection with both of the detecting device and the lifting device, and the lifting device comprises a pallet front support configured to support an extended portion of the pallet, and the pallet front support is movable up and down; and the controller is pre-stored with a predetermined value of the degree of sagging, and in a case that a detected value of the degree of sagging is smaller than the predetermined value, the controller is configured to end the procedure; and in a case that the detected value of the degree of sagging is not smaller than the predetermined value, the controller is configured to determine an adjustment amount of the pallet according to the detected value of the degree of sagging, and then control the lifting device to lift the pallet front support to lift the pallet according to the adjustment amount.

6. The apparatus according to claim 5, comprising a frame having an upper frame, wherein the pallet is slidably connected to the upper frame, and after the pallet slides to a position corresponding to the scanning cross section, a tail portion of the pallet is fixedly connected to the upper frame.

7. The apparatus according to claim 6, wherein the pallet front support is a carrier roller supported on the upper frame with its two ends, and the carrier roller is rollable up and down.

8. The apparatus according to claim 5, wherein the lifting device further comprises a link mechanism connected to a bottom end of the upper frame, and the link mechanism is swingable in a front-rear direction to drive the upper frame to move in a frontward and upward direction or a rearward and downward direction.

9. The apparatus according to claim 6, wherein the lifting device further comprises a link mechanism connected to a bottom end of the upper frame, and the link mechanism is swingable in a front-rear direction to drive the upper frame to move in a frontward and upward direction or a rearward and downward direction.

10. The apparatus according to claim 7, wherein the lifting device further comprises a link mechanism connected to a bottom end of the upper frame, and the link mechanism is swingable in a front-rear direction to drive the upper frame to move in a frontward and upward direction or a rearward and downward direction.

11. The apparatus according to claim 5, wherein the frame has a base seat, the lifting device further comprises two connecting plates connected between the upper frame and the base seat, and the two connecting plates extend right and left and are parallel to each other, each of the connecting plate has two ends hinged to the upper frame and the base seat respectively, and hinged shafts extend right and left and are parallel to each other.

12. The apparatus according to claim 6, wherein the frame has a base seat, the lifting device further comprises two connecting plates connected between the upper frame and the base seat, and the two connecting plates extend right and left and are parallel to each other, each of the connecting plate has two ends hinged to the upper frame and the base seat respectively, and hinged shafts extend right and left and are parallel to each other.

13. The apparatus according to claim 7, wherein the frame has a base seat, the lifting device further comprises two connecting plates connected between the upper frame and the base seat, and the two connecting plates extend right and left and are parallel to each other, each of the connecting plate has two ends hinged to the upper frame and the base seat respectively, and hinged shafts extend right and left and are parallel to each other.

14. A patient table, comprising an apparatus for compensating sagging of a pallet of the patient table, the apparatus comprises a detecting device configured to detect a degree of sagging of a position on the pallet where a scanning cross section is located; a lifting device configured to lift or lower the pallet; and a controller which is in signal connection with both of the detecting device and the lifting device, and the controller is pre-stored with a predetermined value of the degree of sagging, and in a case that a detected value of the degree of sagging is smaller than the predetermined value, the controller is configured to end the procedure; and in a case that the detected value of the degree of sagging is not smaller than the predetermined value, the controller is configured to determine an adjustment amount of the pallet according to the detected value of the degree of sagging, and then control the lifting device to lift the pallet according to the adjustment amount.

15. The patient table according to claim 14, wherein the apparatus comprises a frame having an upper frame, wherein the pallet is slidably connected to the upper frame, and after the pallet slides to a position corresponding to the scanning cross section, a tail portion of the pallet is fixedly connected to the upper frame; and the lifting device comprises a pallet front support configured to support an extended portion of the pallet, and the pallet front support is movable up and down.

16. The patient table according to claim 15, wherein the pallet front support is a carrier roller supported on the upper frame with its two ends, and the carrier roller is rollable up and down.

17. The patient table according to claim 15, wherein the lifting device further comprises a link mechanism connected to a bottom end of the upper frame, and the link mechanism is swingable in a front-rear direction to drive the upper frame to move in a frontward and upward direction or a rearward and downward direction.

18. The patient table according to claim 15, wherein the frame has a base seat, the lifting device further comprises two connecting plates connected between the upper frame and the base seat, and the two connecting plates extend right and left and are parallel to each other, each of the connecting plate has two ends hinged to the upper frame and the base seat respectively, and hinged shafts extend right and left and are parallel to each other.
